SEQUENCE LISTING (4 sequences)

SEQ ID NO: 1 - DNA, 21 bp, Artificial Sequence
  PCR primer to amplify a portion of GFP gene
caccctcgtgaccaccctgac

SEQ ID NO: 2 - DNA, 24 bp, Artificial Sequence
  PCR primer to amplify a portion of GFP gene
caccttgatgccgttcttctgctt

SEQ ID NO: 3 - DNA, 24 bp, Artificial Sequence
  PCR primer to amplify a portion of neor gene
gcttgatccggctacctgcccatt

SEQ ID NO: 4 - DNA, 24 bp, Artificial Sequence
  PCR primer to amplify a portion of neor gene
gccacagtcgatgaatccagaaaa

(19) United States
(12) Patent Application Publication
(10) Pub. No.: US 2004/0214303 A1
(43) Pub. Date: Oct. 28, 2004

(54) Complex

(76) Inventors: Hideto Chono, Moriyama-shi Shiga (JP); Hajime Matsumura, Otsu-shi Shiga (JP); Mitsuhiro Ueno, Kusatsu-shi Shiga (JP); Ikunoshin Kato, Uji-shi Kyoto (JP)

Correspondence Address: BROWDY AND NEIMARK, P.L.L.C., 624 NINTH STREET, NW, SUITE 300, WASHINGTON, DC, 20001-5303, US

(21) Appl. No.: 10/332,704

(22) PCT Filed: Sep. 18, 2001
(86) PCT No.: PCT/JP01/08090

(30) Foreign Application Priority Data
Sep. 20, 2000 (JP) 2000-285857

(51) Int. Cl. C12N 1/20

(52) U.S. Cl. 435/252.3

(57) ABSTRACT

A complex comprising a double-stranded DNA containing a terminal repeated sequence originating in a retrovirus and a DNA having a base sequence which does not occur in this retrovirus and a retrovirus-origin protein component.

### TECHNICAL FIELD

The present invention relates to a method of transduction by transferring a foreign gene into a eukaryote and a series of techniques connected therewith which are useful in fields of medicine, pharmacology, studies of agriculture, forestry and fishery as well as sitology.

### BACKGROUND ART

A method using a virus vector, a method transferring a naked DNA by means of endocytosis, electroporation or gene gun and the like are known as a method for transferring a gene into a eukaryote. Virus vectors are utilized in a field of gene therapy for a wide range of studies including basic and clinical studies. For example, an adenovirus vector is suitable for transient expression of a gene of interest in a target cell in large quantities. A retrovirus vector enables stable long-term expression due to its function of stable integration into a host chromosome. Thus, it is a hopeful vector in a field of gene therapy for genetic diseases and in a field of production of transgenic animals.

However, since gene transfer is mediated by viral infection according to a gene transfer method in which a virus vector is used, the tropism of the virus raises an issue. Specifically, one cannot transfer a gene into a cell that is not expressing a receptor for the virus on its surface. A pseudotype retrovirus vector has been developed in order to overcome this drawback. The pseudotype retrovirus vector utilizes an envelope glycoprotein from vesicular stomatitis virus, VSV-G, and can infect a wide range of hosts. This vector can be used to transfer a gene not only into mammals but also into fishes, birds, insects, amphibians and the like. Attempts have been made to apply the vector to production of transgenic animals. However, this vector has problems because one can transfer only one copy into a target cell due to the nature of a retrovirus, and because it requires at least seven hours from infection to integration into a chromosome.

In case of gene transfer using a naked DNA, the DNA can be readily prepared in large quantities using Escherichia coli or the like, and a gene can be transferred, for example, by means of endocytosis mediated by calcium phosphate transfection or a cationic liposome transfection, electroporation, gene gun or the microinjection technique. In this case, some of the DNAs are integrated into chromosomes through recombination, although the efficiencies are quite low. Therefore, in most cases, these techniques are utilized only when transient gene expression is expected, and are unsuitable for stable long-term expression. Nevertheless, a technique in which a naked DNA is transferred by means of microinjection or the like is exclusively employed for production of transgenic organisms at present. Since integration of a gene into a chromosome is achieved only by accidental recombination as described above, the efficiency of production of a transgenic organism is quite low. Therefore, the production costs a lot and takes a lot of time under the present conditions.

### OBJECTS OF INVENTION

The main object of the present invention is to provide a method for transducing a wide range of cells by transferring a gene regardless of host range utilizing a function of stable integration into a chromosome, which is characteristic of a retrovirus vector, as well as a composition for gene transfer and a composition for transduction used for said method. Thereby, it is possible to establish a technique by which a wide variety of transgenic organisms can be produced and to develop a novel vector in a field of gene therapy.

### SUMMARY OF INVENTION

As a result of intensive studies, the present inventors have prepared a recombinant pre-integration complex (rPIC), transferred it into a target cell, and surprisingly found that the a gene contained in the rPIC is integrated into the chromosome of the target cell regardless of the tropism of the original virus. The rPIC is formed immediately before a recombinant retrovirus vector is integrated into a host chromosome.

Furthermore, the present inventors have developed a complex which can be used to transfer a gene into a wide range of target cells, a method for producing the same, a method for transferring a gene into a cell and a method for transducing a cell using the complex based on the above-mentioned finding. Thus, the present invention has been completed.

The present invention is outlined as follows. The first aspect of the present invention relates to a complex which comprises a double-stranded DNA containing a long terminal repeat derived from a retrovirus and a DNA having a nucleotide sequence that is absent in the retrovirus as well as a protein component derived from a retrovirus.

The second aspect of the present invention relates to a method for producing the complex of the first aspect.

The third aspect of the present invention relates to a gene transfer method, the method comprising transferring the complex of the first aspect into a cell.

The fourth aspect of the present invention relates to a gene transfer method, the method comprising:

transferring, into a cell, a protein component derived from a retrovirus as well as a double-stranded DNA containing a long terminal repeat derived from a retrovirus and a DNA having a nucleotide sequence that is absent in the retrovirus.

The fifth aspect of the present invention relates to a composition for gene transfer, which contains the complex of the first aspect.

The sixth aspect of the present invention relates to a kit for gene transfer, which is used for the gene transfer method of the third or fourth aspect.

The seventh aspect of the present invention relates to a product of a reagent for transferring a gene into a cell,

which contains a packing material and a reagent for gene transfer enclosed in the packing material,

wherein the reagent for gene transfer contains the complex of the first aspect and/or a reagent for preparing the complex, and

wherein it is indicated in a label stuck to the packing material or instructions attached to the packing material that the reagent for gene transfer can be used for gene transfer that does not comprise a step of infecting a cell of interest with a recombinant retrovirus.

The eighth aspect of the present invention relates to a product of a reagent for transferring a gene into a cell,

which contains a packing material and a reagent for gene transfer enclosed in the packing material, wherein the reagent for gene transfer contains a reagent used for the gene transfer method of the fourth aspect, and

wherein it is indicated in a label stuck to the packing material or instructions attached to the packing material that the reagent for gene transfer can be used for gene transfer that does not comprise a step of infecting a cell of interest with a recombinant retrovirus.

The ninth aspect of the present invention relates to a method for transducing a cell, the method comprising transferring the complex of the first aspect into a cell.

The tenth aspect of the present invention relates to a method for transducing a cell, the method comprising transferring a gene by the method of the fourth aspect.

The eleventh aspect of the present invention relates to a composition for transducing a cell, which contains the complex of the first aspect.

The twelfth aspect of the present invention relates to a kit used for transduction of a cell that comprises the gene transfer method of the third or fourth aspect.

The thirteenth aspect of the present invention relates to a product of a reagent for transducing a cell,

which contains a packing material and a reagent for transduction enclosed in the packing material,

wherein the reagent for transduction contains the complex of the first aspect and/or a reagent for preparing the complex, and

wherein it is indicated in a label stuck to the packing material or instructions attached to the packing material that the reagent for transduction can be used for transduction that does not comprise a step of infecting a cell of interest with a recombinant retrovirus.

The fourteenth aspect of the present invention relates to a product of a reagent for transducing a cell,

which contains a packing material and a reagent for transduction enclosed in the packing material,

wherein the reagent for transduction contains a reagent used for the transduction method of the tenth aspect, and

wherein it is indicated in a label stuck to the packing material or instructions attached to the packing material that the reagent for transduction can be used for transduction that does not comprise a step of infecting a cell of interest with a recombinant retrovirus.

### DETAILED DESCRIPTION OF THE INVENTION

The present invention is described in detail below.

The complex of the present invention is a complex for transferring a gene into a target cell. Specifically, it is a complex that comprises at least a double-stranded DNA containing a long terminal repeat (LTR) derived from a retrovirus and a DNA having a nucleotide sequence that is absent in the retrovirus (i.e., a DNA that is of origin different from the retrovirus) as well as a protein component derived from a retrovirus. The complex does not have a structure as a retrovirus. Specifically, the complex of the present invention is not infectious because it does not have an RNA genome and an envelope of a retrovirus, or because its retroviral RNA genome and envelope lacks an ability of infection. In one embodiment, the long terminal repeat derived from a retrovirus and the DNA having a nucleotide sequence that is absent in the retrovirus are derived from a recombinant retrovirus. By using the complex, a gene contained in the double-stranded DNA derived from a recombinant retrovirus, or the DNA having a nucleotide sequence that is absent in the retrovirus, in the complex can be transferred into a target cell. As used herein, a recombinant retrovirus refers to a retrovirus in which a nucleotide sequence of a naturally occurring retroviral genome is partially modified. For example, recombinant retroviruses include a retrovirus into which a foreign gene is inserted, and a retrovirus in which a gene on the viral genome or a portion thereof is subjected to deletion, substitution, insertion or addition.

There is no specific limitation concerning the recombinant retrovirus. It may be ecotropic or amphotropic, or of a pseudotype. For example, a commercially available recombinant retrovirus vector can be used.

There is no specific limitation concerning the double-stranded DNA containing a long terminal repeat (LTR) derived from a retrovirus and a DNA having a nucleotide sequence that is absent in the retrovirus. Preferably, the double-stranded DNA contains two LTRs derived from a retrovirus and a DNA having a nucleotide sequence that is absent in the virus being inserted between the LTRs.

There is no specific limitation concerning the DNA having a nucleotide sequence that is absent in the virus. Any DNA of which the transfer into a cell is desired may be used. For example, a DNA that contains a gene encoding a protein such as an enzyme or a growth factor, or a DNA that contains a gene encoding an antisense nucleic acid, a ribozyme or the like may be used.

The DNA may contain an appropriate marker gene which enables selection of a cell having a gene being transferred. For example, a drug resistance gene which confers resistance to antibiotics on a cell, a reporter gene which enables distinction of a cell having a gene being transferred based on an enzymatic activity or the like may be utilized as a marker gene.

According to the present invention, a gene to be transferred into a cell may be contained in the double-stranded DNA in the complex of the present invention under control of an appropriate promoter such as an LTR promoter in a retrovirus vector or a foreign promoter. Furthermore, the double-stranded DNA may include another regulatory element that cooperates with a promoter or a transcription initiation site (e.g., an enhancer sequence or a terminator sequence) in order to accomplish efficient transcription of a foreign gene. In addition, the double-stranded DNA may include an insulator sequence or the like in order to avoid chromosomal position effect. The foreign gene to be transferred may be a naturally occurring one or an artificially prepared one. Alternatively, it may be obtained by connecting DNA molecules of different origins by a known means such as ligation.

As used herein, a protein component derived from a retrovirus refers to one that has an activity of integrating a viral DNA into a host DNA. Although it is not intended to limit the present invention, for example, a protein component contains an enzyme having an activity of integrating a DNA having a long terminal repeat derived from a retrovirus into a chromosome (e.g., an integrase derived from a retrovirus). Furthermore, the protein component derived from a retrovirus may contain another protein known to be contained in a pre-integration complex (PIC) which is formed before the integration of a gene into a chromosome by a retrovirus. Such a proteins is exemplified by a reverse transcriptase, a nucleic acid-binding protein, a capsid protein or a matrix protein. Also, intracellular factors such as a non-histone protein HMG-1 or BAF-1, or a DNA-dependent protein kinase (DNA-PK) may be included. The protein component derived from a retrovirus used according to the present invention may have deletion, substitution, insertion or addition in a naturally occurring protein component derived from a retrovirus as long as it has an activity of integrating a viral DNA into a host chromosome.

A retrovirus infects a dividing and growing cell, and its viral gene is integrated into a chromosome of a host cell. Integration of a gene into a chromosome by a retrovirus is achieved through a series of steps as follows: (1) attachment of a viral particle to a surface of a target cell via a receptor; (2) release of a core for invasion into a cytoplasm; (3) synthesis of a cDNA by a reverse transcriptase in the core; (4) synthesis of a double-stranded DNA utilizing an RNase H activity and a DNA-dependent DNA synthesis activity of the enzyme; (5) formation of a pre-integration complex (PIC); (6) transport of the PIC into a nucleus; and (7) stable integration into a host chromosome by action of an enzyme having an integration activity.

Transport of a PIC into a nucleus of a host through breakdown of a nuclear membrane of a host cell is required for integration of a gene into a chromosome. The transport is achieved by passage through the G2/M phase of the host cell. Thus, the PIC cannot be transported into a nucleus in a non-dividing cell, and integration of a gene into the chromosome does not occur. Therefore, if a cell infected with a retrovirus is arrested at the G1 phase, the PIC that cannot be transported into a nucleus is accumulated in a cytoplasm. One can prepare the accumulated PIC from the cytoplasm.

For example, the complex of the present invention can be preferably prepared as follows utilizing the above-mentioned mechanism.

A cell arrested at the G1 phase by treatment with a cell cycle-regulating agent such as aphidicolin is infected with a recombinant retrovirus. The infected cell is cultured. As used herein, culturing is not limited to incubation of a cell for increasing the number of the cell, but includes maintenance of a cell in a state of arrest cell cycle. Reverse transcription, synthesis of a double-stranded DNA and formation of a recombinant pre-integration complex (rPIC) proceed in a cytoplasm during a period of seven to eleven hours from infection. Since the cell cycle is arrested at the G1 phase, the produced rPIC is not integrated into a chromosome, but accumulated in the cytoplasm. The rPIC accumulated in the cytoplasm can be readily extracted by degrading a cell membrane with a detergent. The extracted rPIC can be used in the method of the present invention. Furthermore, the extracted rPIC can be purified by a known method such as centrifugation or chromatography. The thus obtained purified rPIC can be also used in the method of the present invention.

The cultured cell used for the preparation of the complex of the present invention may be any cell that can be infected with the retrovirus to be used. For example, NIH/3T3 cell (ATCC CRL-1658), HT-1080 cell (ATCC CCL-121) or 293 cell (ATCC CRL-1573) can be used.

It is possible to efficiently infect a cell arrested at the G1 phase with a recombinant retrovirus by using an appropriate supplementary agent such as polybrene or a functional substance as described in WO 95/26200 or WO 97/18318. A fibronectin fragment can be used as such a functional substance. Particularly, RetroNectin (Takara Shuzo) can be preferably used.

A reverse transcriptase activity and a DNA synthase activity, which are necessary for synthesis of a double-stranded DNA, as well as a protein component (e.g., an integrase), which is necessary for integration into a chromosome, are present in a recombinant retroviral particle. Thus, the complex of the present invention can be also prepared by adding deoxyribonucleotides to a recombinant retroviral particle destroyed in vitro and incubating the mixture.

Specifically, a recombinant retroviral particle is destroyed in vitro under appropriate conditions. Deoxyribonucleotides are added thereto. Then, cDNA synthesis and a reaction of synthesis of a double-stranded DNA proceed in vitro. The synthesized double-stranded DNA is associated with a protein component (e.g., an integrase). Thereby, an rPIC is formed. The rPIC formed as described above can be used in the method of the present invention. Furthermore, the formed rPIC can be purified by a known method such as centrifugation or chromatography. The thus obtained purified rPIC can be also used in the method of the present invention.

Also, the complex of the present invention can be prepared without the use of a viral particle by preparing a double-stranded DNA containing an LTR derived from a retrovirus and a nucleotide sequence that is absent in the virus, and a protein component derived from a retrovirus independently, and mixing them together in vitro.

There is no specific limitation concerning the double-stranded DNA containing an LTR derived from a retrovirus and a DNA having a nucleotide sequence that is absent in the virus to be used. For example, the double-stranded DNA contains two LTRs derived from a retrovirus and a DNA having a nucleotide sequence that is absent in the virus being inserted between the LTRs. There is no specific limitation concerning the size of the DNA having a nucleotide sequence that is absent in the virus. A DNA of any size can be used depending on the object.

There is no specific limitation concerning the method for preparing a double-stranded DNA containing an LTR derived from a retrovirus and a DNA having a nucleotide sequence that is absent in the virus. For example, a plasmid having a cassette of an LTR derived from a retrovirus and a DNA having a nucleotide sequence that is absent in the retrovirus is constructed, a host cell (e.g., Escherichia coli) transformed with the plasmid is cultured, and the double-stranded DNA can be prepared in large quantities from the plasmid isolated from the culture. If the plasmid is constructed such that cleavage sites for a restriction enzyme (or restriction enzymes) are placed in on both sides of the double-stranded DNA containing an LTR derived from a retrovirus and a DNA having a nucleotide sequence that is absent in the virus, the double-stranded DNA of interest can be readily prepared by allowing the restriction enzyme(s) to act. The double-stranded DNA containing an LTR derived from a retrovirus and a DNA having a nucleotide sequence that is absent in the virus can be also prepared by amplification using the plasmid having the cassette as a template by a gene amplification method such as a PCR.

There is no specific limitation concerning the method for preparing the protein component derived from a retrovirus to be used according to the present invention. For example, it may be prepared from a viral particle, by genetic engineering or from a packaging cell. The protein component derived from a retrovirus to be used according to the present invention (e.g., an integrase) can be readily prepared by cloning a gene encoding the component into an expression vector, and expressing the gene by genetic engineering using the expression vector and Escherichia coli or the like as a host.

A culture supernatant obtained by culturing a packaging cell contains a component having a DNA integration activity. Therefore, the protein component derived from a retrovirus used according to the present invention can be prepared from the culture supernatant. For example, a protein complex containing the component can be prepared by subjecting a culture supernatant obtained by culturing a packaging cell to ultracentrifugation. Alternatively, a protein complex containing the component can be obtained from an extract of a packaging cell. The protein complex can be used according to the present invention. Furthermore, a component having a DNA integration activity can be purified from the protein complex by a known method. The thus obtained purified component can be also used according to the present invention.

There is no specific limitation concerning the packaging cell to be used. For example, BOSC23 cell or the like is preferably used (Proc. Natl. Acad. Sci. USA, 90:8392-8396 (1993)).

The complex of the present invention can be prepared in vitro without the use of a viral particle by mixing in vitro a double-stranded DNA containing an LTR derived from a retrovirus and a nucleotide sequence that is absent in the virus with a protein component derived from a retrovirus, both of which have been prepared independently in vitro as described above. The complex prepared as described above can be used in the method of the present invention. Furthermore, the complex can be optionally purified by a known method such as centrifugation or chromatography. The purified complex can be also used in the method of the present invention.

As described above, there is no specific limitation concerning the size of the double-stranded DNA in the complex of the present invention if the complex is prepared in vitro. Therefore, by using the complex, it is possible to transfer a gene of 8 kb or longer which could not be transferred according to a conventional gene transfer method using a retrovirus vector.

The complex may include another element as long as transfer of the complex into a cell and integration of a DNA into a chromosome are not prevented.

The complex of the present invention need not be packaged in a retroviral particle.

The complex of the present invention may be used for gene transfer immediately after preparation. Alternatively, it may be stored by freezing and used after thawing upon use. Although there is no specific limitation concerning the cryopreservation method, the complex may be rapidly frozen using dry ice and then stored at −80° C., for example.

By transferring the complex prepared as described above into a target cell, a gene can be transferred into a chromosome of the target cell, or the target cell can be transduced.

There is no specific limitation concerning the method for transferring the complex into a target cell. For example, the complex can be transferred by a known method such as a method in which endocytosis (e.g., a cationic liposome method or a calcium phosphate method), or a perforation method such as electroporation, gene gun or microinjection is utilized. If the complex is transferred using a perforation method, a transduced cell can be efficiently obtained by culturing a target cell in the presence of a substance having a cell adhesion activity after transfer as described in WO 96/17073.

By transferring, into a target cell, a double-stranded DNA containing an LTR derived from a retrovirus and a nucleotide sequence that is absent in the virus as well as a protein component derived from a retrovirus, a gene can be transferred into a chromosome of the target cell, or the target cell can be transduced.

There is no specific limitation concerning the order of the transfer of the double-stranded DNA containing an LTR derived from a retrovirus and a nucleotide sequence that is absent in the virus and the transfer of the protein component derived from a retrovirus. The double-stranded DNA containing an LTR derived from a retrovirus and a nucleotide sequence that is absent in the virus may be transferred first, or the protein component derived from a retrovirus may be transferred first. Alternatively, they may be transferred at the same time. There is no specific limitation concerning the method for transferring the double-stranded DNA containing an LTR derived from a retrovirus and a nucleotide sequence that is absent in the virus as well as the protein component derived from a retrovirus into a target cell. For example, they can be transferred by a known method such as a method in which endocytosis is utilized (e.g., a cationic liposome method or a calcium phosphate method) or a perforation method such as electroporation, gene gun or microinjection. If the transfer is carried out using a perforation method, a transduced cell can be efficiently obtained by culturing a target cell in the presence of a substance having a cell adhesion activity after transfer as described in WO 96/17073.

There is no specific limitation concerning the origin of a target cell used for the gene transfer method or the transduction method of the present invention. A wide variety of eukaryotic cells can serve as targets without restrictions due to the tropism of the recombinant retrovirus from which the complex of the present invention is derived. For example, a cell from a eukaryote such as a mammal, a bird, a fish, an insect or a plant can be used as a target.

There is no specific limitation concerning the type of the target cell. All kinds of cells (including, for example, stem cells such as a hematopoietic stem cell and an embryonic stem cell, germ cells such as an egg and a sperm, and various somatic cells) can serve as targets.

The target cell may be a cultured cell or an individual of an organism. A transgenic animal can be produced by using a fertilized egg or a germ line from a vertebrate as a target.

A conventional gene transfer using a retrovirus vector requires at least seven hours from infection with a virus to integration of a gene into a chromosome. This is because formation of a pre-integration complex in a cytoplasm requires time. The target cells divide during the time, resulting in a mosaic in which cells with the transferred gene and cells without the transferred gene are generated. This phenomenon has been a great problem about production of transgenic animals.

On the other hand, according to the gene transfer method of the present invention, a gene is integrated into a chromosome immediately after transfer. Therefore, such a mosaic is not formed. Thus, the gene transfer method and the transduction method of the present invention enable efficient production of a transgenic organism.

Furthermore, according to a conventional gene transfer using a retrovirus vector, one can transfer only one copy of a gene in a target cell. On the other hand, plural copies of a gene can be transferred into a target cell by adjusting the amount of the complex to be transferred according to the transfer method of the present invention.

The composition for gene transfer of the present invention and the composition for transduction of the present invention are used for the gene transfer method of the present invention and the transduction method of the present invention, respectively, and contains the complex of the present invention or a protein component derived from a retrovirus. The composition may contain an appropriate buffer, a salt or the like. It may additionally contain any substance that promotes gene transfer.

The kit for gene transfer of the present invention and the kit for transduction of the present invention are used for the gene transfer method of the present invention and the transduction method of the present invention, respectively. The composition is in a packed form, and contains a reagent used for the method as well as instructions that direct transferring, into a cell, of the complex of the present invention, or of a protein component derived from a retrovirus as well as a double-stranded DNA containing a long terminal repeat derived from a retrovirus and a DNA having a nucleotide sequence that is absent in the retrovirus. As used herein, instructions refer to a document that directs a method for carrying out the present invention. For example, the instructions may be a printed matter that describes the gene transfer method of the present invention, the transduction method of the present invention, a method of using the complex of the present invention or the protein component derived from a retrovirus in said method, recommended reaction conditions and the like. The instructions include an instruction manual in a form of a pamphlet or a leaflet, a label stuck to the kit, and description on the package containing the kit. The instructions also include information disclosed or provided through electronic media such as the Internet.

The kit of the present invention may contain a cell used for preparing the complex of the present invention, a medium, a reagent and/or a culture vessel for culturing the cell, a reagent used for transferring, into a target cell, of the complex, a protein component derived from a retrovirus, or a double-stranded DNA containing a long terminal repeat derived from a retrovirus and a DNA having a nucleotide sequence that is absent in the retrovirus.

One can conveniently carry out the gene transfer method and the transduction method of the present invention using the kit.

The product of a reagent for gene transfer or transduction of the present invention is a product which contains a packing material and a reagent for gene transfer or cell transduction enclosed in the packing material, wherein the reagent for gene transfer or transduction contains a complex that comprises a double-stranded DNA containing a long terminal repeat derived from a retrovirus and a DNA having a nucleotide sequence that is absent in the retrovirus as well as a protein component derived from a retrovirus and/or a reagent for preparing the complex, and wherein it is indicated in a label stuck to the packing material or instructions attached to the packing material that the reagent for gene transfer or transduction can be used for gene transfer or transduction that does not comprise a step of infecting a cell of interest with a retrovirus.

Alternatively, the product of a reagent for gene transfer or transduction of the present invention is a product which contains a packing material and a reagent for gene transfer or transduction enclosed in the packing material, wherein the reagent for gene transfer or transduction contains a composition containing a protein component derived from a retrovirus, and wherein it is indicated in a label stuck to the packing material or instructions attached to the packing material that the reagent for gene transfer can be used for gene transfer or transduction that does not comprise a step of infecting a cell of interest with a recombinant retrovirus.

According to the present invention, the drawback associated with a conventional gene transfer method using a retrovirus vector, i.e., restriction on host range, is overcome, and a gene can be transferred into all eukaryotic cells. Specifically, since the conventional gene transfer using a retrovirus vector involves infection via a receptor for the virus, host range is restricted depending on the presence of the receptor. On the other hand, the complex of the present invention can be transferred using a known method such as a method in which endocytosis is utilized, electroporation, gene gun or microinjection regardless of the presence of such a receptor. As a result, a gene can be transferred into a wide variety of cells without restriction on host range. Furthermore, a gene is integrated into a chromosome immediately after transfer into a target cell of interest according to the present invention. Therefore, a mosaic which has been a problem associated with a conventional method for producing a transgenic organism is not formed. A transgenic organism can be efficiently produced.

A complicated operation is required for producing viral particles to be used according to the conventional method for gene transfer using a virus vector. Furthermore, the conventional method has a drawback in that it is difficult to prepare a high-titer virus vector. On the other hand, the complex of the present invention can be prepared in vitro without the use of a viral particle. In this case, the complex can be prepared in large quantities in a short time without a complicated operation. Thus, it is now possible to transfer a gene much more conveniently and efficiently as compared with the conventional method.

As described above, the present invention is an excellent technique which overcomes the drawbacks associated with conventional techniques, and is excellently useful, for example, in that a remarkable therapeutic effect is expected when the present invention is applied to any gene therapy of which the effect has been restricted because of the limitation associated with the conventional methods.

### EXAMPLES

The following Examples illustrate the present invention in more detail, but are not to be construed to limit the scope thereof.

### Example 1

Preparation of recombinant pre-integration complex (rPIC)

1. Preparation of EcoGFP Virus Supernatant

A cell producing a ecoGFP virus was established by introducing a plasmid containing a gene for green fluorescence protein (GFP) and a neomycin-resistance gene into a packaging cell GP+E-86 [Journal of Virology, 62:1120-1124 (1988)]. The cell was cultured in Dulbecco's modified Eagle's medium (DMEM, Sigma) supplemented with 10% fetal calf serum (Bio Whittaker) containing 50 units/ml of penicillin (Gibco) and 50 μg/ml of streptomycin (Gibco) at 37° C. in the presence of 5% CO₂. The DMEM used in procedures as described hereinafter was contained 50 units/ml of penicillin and 50 μg/ml of streptomycin in all cases. A suspension of the ecoGFP virus was prepared as follows. The producer cell was grown to semi-confluence in a 10-cm plate. 7 ml of DMEM supplemented with 10% fetal calf serum was added thereto. After culturing for 24 hours at 32° C. in the presence of 5% CO₂, the culture supernatant was filtered through a 0.45-micron filter (Millipore) to prepare a stock of a viral supernatant, which was stored at −80° C. until use.

2. Determination of titer of viral supernatant

Titer of the viral supernatant was determined according to a standard method [Journal of Virology, 62:1120-1124 (1988)] using NIH/3T3 cell (ATCC CRL-1658) Briefly, 2 ml of DMEM supplemented with 10% calf serum (Gibco) containing 5×10⁴ NIH/3T3 cells was added to each well of a 6-well tissue culture plate (Iwaki Glass). The plate was incubated at 37° C. overnight in the presence of 5% CO₂. The medium was removed by suction. 1 ml of a serial dilution of the viral supernatant and hexadimethrine bromide (polybrene, Aldrich) at a final concentration of 8 ug/ml was added to each well. The plate was incubated at 37° C. for four hours. 1 ml of DMEM supplemented with 10% calf serum was further added thereto, and the plate was incubated for 20 hours. The medium was then exchanged for 2 ml of DMEM supplemented with 10% calf serum containing G418 (Gibco) at a final concentration of 0.5 mg/ml. The incubation was further continued. The medium containing G418 was exchanged at intervals of three to four days. After 10 to 12 days, grown G418-resistant colonies were stained with a 0.2% methylene blue solution in methanol and the number was counted. The number of infectious virus particles in 1 ml of a supernatant (cfu/ml) as the titer of the virus was calculated by multiplying the number of colonies in a well by the dilution ratio of the viral supernatant. The titer of the suspension of the ecoGFP virus prepared as described above was 1×10⁷ cfu/ml.

3. Preparation of rPIC

2×10⁶ NIH/3T3 cells were cultured in a 10-cm plate containing 10 ml of DMEM supplemented with 10% calf serum for 24 hours at 37° C. in the presence of 5% CO₂. The medium was exchanged for 7 ml of DMEM supplemented with 10% calf serum containing 2 μg/ml of aphidicolin (Wako Pure Chemical Industries). The plate was incubated for additional 14 hours at 37° C. in the presence of 5% CO₂. The medium was removed by suction fourteen hours after the addition of aphidicolin. A mixture prepared by mixing 2 ml of the suspension of the ecoGFP virus with 3 ml of DMEM supplemented with 10% calf serum, and adding aphidicolin and polybrene at final concentrations of 2 μg/ml and 8 μg/ml, respectively, was added to the NIH/3T3 cells to start infection with the virus at 32° C. in the presence of 5% CO₂. The viral suspension was removed three hours after the start of infection, and the medium was exchanged for 7 ml of DMEM supplemented with 10% calf serum containing 2 μg/ml of aphidicolin. The plate was incubated for additional seven hours (for 10 hours from the start of viral infection) at 32° C. in the presence of 5% CO₂. During this period, reverse transcription and DNA synthesis reactions proceeded to form rPICs in the NIH/3T3 cells infected with the virus. However, since the cell cycle of the NIH/3T3 cell was arrested at the G1 phase by the action of aphidicolin, the rPICs were accumulated in the cytoplasm without integrating the retroviral gene into the chromosome.

The rPICs accumulated in the cytoplasm were extracted as follows. Briefly, cell were washed with 5 ml of PBS 10 hours after infection and dispersed using 2 ml of trypsin-EDTA (Bio Whittaker). 5 ml of DMEM supplemented with 10% calf serum was added thereto. Cells precipitated by centrifugation at 200×g for five minutes were washed with 9 ml of a buffer A (10 mM Tris, 225 mM KCl, 5 mM MgCl₂, 1 mM DTT, 20 μg/ml aprotinin, pH 7.4). A precipitate obtained by centrifugation at 200×g for five minutes was suspended in 250 μl of the buffer A. 1.25 μl of a 5% digitonin solution in DMSO was added to the cell suspension. A cytoplasmic fraction was extracted by allowing the mixture to stand at 25° C. for five minutes. The extract was centrifuged at 1000×g for three minutes. The supernatant was further centrifuged at 5000×g for three minutes. 50 μl aliquots of the thus obtained supernatant as an rPIC suspension were dispensed and stored at −80° C.

4. Detection of Double-Stranded DNA

The GFP gene derived from the recombinant retrovirus was detected by a PCR using a dilution of the rPIC suspension obtained by extraction and cryopreserved as described in 3 above as a template and GFP gene-specific primers. A PCR reaction mixture prepared by mixing 0.5 μl of TaKaRa Taq (5 units/μl, Takara Shuzo), 5 μl of 10×PCR buffer (Takara Shuzo), 8 μl of 1.25 mM DNTP mix, 1 μl of the template, 20 pmol of Primer 1 (SEQ ID NO:1), 20 pmol of Primer 2 (SEQ ID NO:2) and distilled water to a volume of 50 μl was overlaid with mineral oil, and subjected to heating at 94° C. for 1 minute followed by a reaction of 30 cycles each cycle consisting of 94° C. for 30 seconds, 59° C. for 30 seconds and 72° C. for 30 seconds. After reaction, 10 μl of the PCR reaction mixture was subjected to electrophoresis on 2% agarose gel to confirm an amplified fragment. A dilution of the rPIC or a DNA purified from the rPIC by phenol-chloroform extraction was used as a template. A fraction extracted from NIH/3T3 cells without viral infection in a similar manner was subjected to a PCR as a negative control. An expression plasmid vector for GFP was used as a positive control. As a result, amplification of a 316-bp fragment derived from the GFP gene was observed when the dilution of the rPIC or the DNA purified from the rPIC by phenol-chloroform extraction was used as a template. Therefore, it was shown that the rPIC prepared as described in 3 above contained the GFP gene derived from the recombinant virus.

### Example 2

Transfer of rPIC into cultured cell

Mouse NIH/3T3 cells and human 293 cells (ATCC CRL-1573) were cultured at 37° C. in the presence of 5% CO₂ using DMEM supplemented with 10% calf serum and DMEM supplemented with 10% fetal calf serum as growth media, respectively. The mouse NIH/3T3 cells and human 293 cells grown to confluence in 10-cm plates were detached from the plates by trypsinization. The respective cells were washed with serum-free DMEM and suspended in 700 μl of DMEM. 700 μl of the cell suspension and 100 μl of the rPIC prepared in Example 1 were placed in a 0.4-cm width electroporation cuvette (Bio-Rad) and subjected to electroporation using Gene Pulser II (Bio-Rad) at 250 V, 975 μF and room temperature. A 1/10 volume for mouse NIH/3T3 cells or a ¼ volume for human 293 cells was seeded into a 10-cm gelatin-coated plate (Iwaki Glass). The cells were cultured in the growth medium containing 0.5 mg/ml of G418 starting from the day after electroporation.

Cells were observed under a fluorescence microscope seven days after electroporation. Cells emitting green fluorescence were observed for both mouse NIH 3T3 cells and human 293 cells. Thus, it was shown that the transferred GFP gene was expressed in cells derived from both a mouse and a human.

Neomycin (G418)-resistant colonies were formed when the cultivation was further continued for two weeks after the electroporation in a G418 selection medium. The colonies were stained with methylene blue. 46 resistant colonies and 196 resistant colonies were observed for mouse NIH/3T3 cells and human 293 cells, respectively. Thus, it was shown that the transferred gene was stably expressed.

The above-mentioned results show that an rPIC prepared from an ecotropic virus which can infect only cells derived from mice could be used to stably transfer a gene into cells derived from humans into which such a gene could not have been transferred according to a conventional method, and that the rPIC is a vector that can be used to stably transfer a gene regardless of host range.

### Example 3

Preparation of high-titer rPIC and assessment of integration efficiency of rPIC

1. Preparation of High-Titer rPIC

A high-titer ecoGFP virus suspension was prepared by centrifuging 250 ml of the ecoGFP virus supernatant at a low speed (2900×g) for 16 hours at 4° C., and suspending the precipitate in 5 ml of DMEM supplemented with 10% calf serum. The titer of the viral suspension was 4×10⁸ cfu/ml. The viral suspension was used to infect NIH/3T3 cells at M.O.I. of 200. High-titer rPICs were prepared as described in Example 2.

2. Assessment of Integration Efficiency of rPIC

Gene transfer into 293 cells was carried out by electroporation using the high-titer rPIC. As controls, gene transfers into 293 cells were carried out a by electroporation or transfection using a plasmid vector containing the GFP gene and the neomycin-resistance gene. Three days after gene transfer, 293 cells expressing GFP were selectively recovered using a flow cytometer FACS Vantage (Becton Dickinson) . The ratios of GFP-expressing cells of the recovered 293 cells were 94.3% (transfer of the rPIC), 94.3% (transfer of the plasmid vector by electroporation) and 98.8% (transfer of the plasmid vector by transfection), indicating that cells with the gene transiently transferred were selectively recovered. The 293 cells transiently expressing GFP were cultured in a G418+ medium or a G418− medium for two weeks. An efficiency of stable long-term gene expression as an integration efficiency was calculated according to the following equation: [number of colonies in a plate with G418]÷[number of colonies in a plate without G418]. The integration efficiencies were 98.7% (transfer of the rPIC), 7.5% (transfer of the plasmid vector by electroporation) and 1.4% (transfer of the plasmid vector by transfection), indicating that the rPIC is a vector that results in an excellent integration efficiency.

### Example 4

Transfer of rPIC into fertilized egg of medaka fish

A pair of mature medaka fish (Oryzias latipes, orange-red variety) consisting of a male and a female was bred in 3 L of tap water at 25° C. under conditions of a light period for 14 hours and a dark period for 10 hours. They were fed with. TetraFin (TetraWerke) three to five times a day setting the amount of TetraFin such that it was consumed within three to five minutes, and then spawned. Eggs were collected immediately after spawning, separated each other using tweezers in a 8-ppm aqueous solution of methylene blue and washed with the aqueous solution. The high-titer rPIC prepared in Example 3 was purified on a gel filtration column Superose 6 (Amersham Pharmacia). 5 μl of a fraction containing the rPIC was placed in a microinjection pipette (prepared using a capillary tubing model GD-1, Narishige), and about 50 pl of the rPIC suspension was injected into each one-cell stage medaka fertilized egg under a microscope. 69 eggs were subjected to microinjection, placed in a 96-well plate such that each well contained one egg, and cultured in the 8-ppm aqueous solution of methylene blue at 25° C. Hatched fry eight days after spawning or thereafter were transferred one by one to a fish tank. Finally, a total of 47 fry hatched. 16 fry were sampled immediately after spawning, placed in 1.5-ml microtubes and stored at −20° C. A genomic DNA was extracted from a hatched fry as follows and subjected to detection of the neomycin-resistance gene by a PCR. Briefly, 100 μl of an extraction buffer (10 mM Tris, 100 mM NaCl, 10 mM EDTA, 39 mM DTT, 2% SDS, 50 μg/ml Proteinase K, pH 8) was added to each microtube containing one fry. The tube was incubated at 60° C. for four hours. The tube was stirred 30 and 60 minutes after the start of the incubation. 5 μg of RNase A was added per tube, and the tube was incubated at 37° C. for an hour. A genomic DNA obtained after phenol-chloroform extraction and ethanol precipitation was dissolved in 50 μl of distilled water.

The transferred gene was detected by a PCR using 1 μl of the thus obtained genomic DNA solution as a template and primers specific for the neomycin-resistance gene. TaKaRa Taq (Takara Shuzo) was used for the reaction. A mixture of 0.5 μl of TaKaRa Taq, 5 μl of 10×PCR buffer, 8 μl of dNTP mix, 1 μl of the diluted template, 20 pmol of Primer 3 (SEQ ID NO:3), 20 pmol of Primer 4 (SEQ ID NO:4) and distilled water to a volume of 50 μl was overlaid with mineral oil, and subjected to heating at 94° C. for 1 minute followed by a reaction of 30 cycles each cycle consisting of 94° C. for 30 seconds, 56° C. for 30 seconds and 72° C. for 30 seconds. 10 μl of the PCR reaction mixture was subjected to electrophoresis on 2% agarose gel to confirm an amplified fragment. As a result, the transferred gene was detected in 16 out of 16 medaka fishes into which the rPIC was transferred by microinjection. Thus, the efficiency of the gene transfer was 100%. A fertilized egg of medaka fish is at the one-cell stage for an hour after fertilization. It divides once in 35 minutes thereafter. If a VSV-G retrovirus vector is used for a cell dividing so rapidly, a gene is integrated only at an advanced developmental stage because the reverse transcription process limits the rate. Furthermore, a plasmid vector is integrated into a host chromosome only by accidental recombination. On the other hand, since the rPIC is extracted immediately before integration into the genome of NIH/3T3 cell, it is prepared in a state that results in a high integration efficiency. Therefore, integration into a genome at an early stage of cell division is expected. The results show that the rPIC is a vector that can be applied to a wide variety of subjects, for example, as a vector for producing a transgenic animal.

### Example 5

### Gene Transfer Into Cultured Cells Utilizing Supernatant of BOSC23 Cell

A packaging cell BOSC23 has retroviral gag-pol genes and an ecotropic env gene being transferred. BOSC cell was cultured in Dulbecco's modified Eagle's medium supplemented with 10% fetal calf serum containing 50 units/ml of penicillin and 50 μg/ml of streptomycin at 37° C. in the presence of 5% CO₂.

BOSC23 cell was grown to semi-confluence in a 10-cm plate. The medium was exchanged for 5 ml of fresh DMEM supplemented with 10% fetal calf serum. Cultivation was carried out for 24 hours at 37° C. in the presence of 5% CO₂.

A supernatant of the culture was filtered through a 0.45-micron filter (Millipore) and centrifuged in a ultracentrifuge at 30,000 rpm at 4° C. for two hours.

The resulting precipitate was washed with DMEM and centrifuged again in a ultracentrifuge at 30,000 rpm at 4° C. for two hours. The resulting precipitate was suspended in 50 μl of DMEM. An SDS-containing reagent was added thereto. The mixture was heated at 99° C. for five minutes, and then subjected to electrophoresis for proteins (SDS-PAGE).

As a result, it was confirmed that the precipitate contained gag proteins (p30, p15).

These results suggested the presence of a protein complex containing geg-pol-env proteins in the culture supernatant of BOSC23 cell. It was expected that the protein complex had an ability to infect cells due to the function of the env protein. Experiments were carried out to examine an ability to integrate a plasmid DNA into a genome of a cultured cell utilizing the protein complex in the culture supernatant of BOSC23 cell.

1. Preparation of BOSC23 Cell Supernatant Sample

A BOSC23 cell supernatant sample was obtained as follows. BOSC23 cell were grown to semi-confluence in a 10-cm plate. The medium was exchanged for 10 ml of fresh DMEM supplemented with 10% fetal calf serum. A culture supernatant obtained after culturing at 32° C. in the presence of 5% CO₂ for 24 hours was filtered through a 0.45-micron filter (Millipore).

2. Gene Transfer Utilizing BOSC23 Cell Supernatant Sample

Mouse NIH/3T3 cells were cultured at 37° C. in the presence of 5% CO₂ using DMEM supplemented with 10% calf serum as a growth medium. A retrovirus vector plasmid, pDON-AI-rsGFP, which contains the GFP gene and the neomycin-resistance gene between retroviral LTR was transfected into mouse NIH/3T3 cells grown to semi-confluence in a 10-cm plate using LipofectAMINE 2000 (Gibco). The cells were cultured in 10 ml of DMEM supplemented with 10% calf serum at 37° C. in the presence of 5% CO₂ for five hours to prepare a test sample. pDON-AI-rsGFP was constructed by incorporating a gene encoding rsGFP prepared from an rsGFP-encoding plasmid pQBI25 (Takara Shuzo) into a retrovirus vector pDON-AI. After culturing for five hours, the medium was removed from the test sample by suction. 4 ml of the BOSC23 cell supernatant sample prepared in 1 above was added thereto. Hexadimethrine bromide (polybrene, Aldrich) was further added at a final concentration of 8 μg/ml. Cultivation was carried out for 32° C. for 16 hours. After culturing for 16 hours, the BOSC23 cell supernatant sample was removed by suction, 10 ml of DMEM supplemented with 10% calf serum was added thereto, and cultivation was carried out at 37° C. in the presence of 5% CO₂ for 24 hours. After culturing for 24 hours, the cells were recovered. 20,000 of the cells were seeded into a 6-cm gelatin-coated plate. The cells were cultured in DMEM supplemented with 10% calf serum containing G418 (Gibco) at a final concentration of 0.5 mg/ml. After 14 days, grown G418-resistant colonies were stained with a 0.2% methylene blue solution in methanol and the number was counted. In addition, as a control, 500 of the cells were seeded into a 6-cm gelatin-coated plate, and cultured in a normal medium without G418 at 37° C. in the presence of 5% CO₂ for 14 days. Then, grown colonies were stained with a 0.2% methylene blue solution in methanol and the number was counted.

As a control, the medium was removed by suction five hours after gene transfer by transfection, a normal medium was added thereto, and cultivation was carried out at 37° C. in the presence of 5% CO₂.

After 40 hours, the cells were recovered. 20,000 of the cells were seeded into a 6-cm gelatin-coated plate. The cells were cultured in DMEM supplemented with 10% calf serum containing G418 (Gibco) at a final concentration of 0.5 mg/ml. After 14 days, grown G418-resistant colonies were stained with a 0.2% methylene blue solution in methanol and the number was counted. In addition, as a control, 500 of the cells were seeded into a 6-cm gelatin-coated plate, and cultured in a normal medium at 37° C. in the presence of 5% CO₂ for 14 days. Then, grown colonies were stained with a 0.2% methylene blue solution in methanol and the number was counted.

An efficiency of long-term gene expression (an integration efficiency) was calculated according to the following equation: [number of colonies in a plate with G418]÷[number of colonies in a plate without G418]. The integration efficiency determined for the test sample was 5.00%, whereas the integration efficiency for the control was 0.76%. These results show that the plasmid vector could be integrated into the genome of the host cell with an efficiency about seven times higher due to the action of the protein complex containing the gag-pol-env proteins in the BOSC23 cell supernatant sample by transferring the plasmid vector into the cell and then infecting the transferred cell with the BOSC23 cell supernatant sample. The results show that the plasmid vector having an LTR and the retroviral structural protein formed a complex in the target cell.

### Industrial Applicability

The present invention provides a method for efficiently transferring a gene regardless of host range. Since a gene transferred according to the gene transfer method of the present invention is immediately integrated into a host chromosome, the method enables efficient production of a transgenic organism.

### Sequence Listing Free Text

SEQ ID NO:1: PCR primer to amplify a portion of GFP gene.

SEQ ID NO:2: PCR primer to amplify a portion of GFP gene.

SEQ ID NO:3: PCR primer to amplify a portion of neo^{r}gene.

SEQ ID NO:4: PCR primer to amplify a portion of neo^{r} gene.

1. A complex which comprises a double-stranded DNA containing a long terminal repeat derived from a retrovirus and a DNA having a nucleotide sequence that is absent in the retrovirus as well as a protein component derived from a retrovirus.

2. The complex according to claim 1, wherein the double-stranded DNA containing a long terminal repeat derived from a retrovirus and a DNA having a nucleotide sequence that is absent in the retrovirus is derived from a recombinant retrovirus.

3. The complex according to claim 2, wherein the recombinant retrovirus is obtainable by infecting a cell susceptible to the recombinant retrovirus with the recombinant retrovirus.

4. The complex according to claim 3, wherein the cell cycle of the cell susceptible to the recombinant retrovirus is arrested.

5. The complex according to claim 1, which is obtainable by mixing an isolated double-stranded DNA containing a long terminal repeat derived from a retrovirus and a DNA having a nucleotide sequence that is absent in the retrovirus with an isolated protein component derived from a retrovirus.

6. The complex according to claim 1, wherein the protein component derived from a retrovirus is derived from a culture supernatant of a packaging cell and/or from an extract of a packaging cell.

7. The complex according to claim 1, wherein the protein component derived from a retrovirus contains an enzyme having an activity of integrating a DNA having a long terminal repeat derived from a retrovirus into a chromosomal DNA.

8. A method for producing a complex, the method comprising:
(a) infecting a cell susceptible to a recombinant retrovirus with the recombinant retrovirus that contains a long terminal repeat derived from a retrovirus and a nucleotide sequence that is absent in the retrovirus;
(b) culturing the cell infected in step (a); and
(c) collecting a complex that comprises a double-stranded DNA derived from the recombinant retrovirus and a protein component derived from the retrovirus from the cell cultured in step (b).

9. The method according to claim 8, wherein the cell cycle of the cell susceptible to a recombinant retrovirus is arrested.

10. A method for producing a complex, the method comprising:
(a) destroying a particle of a recombinant retrovirus that contains a long terminal repeat derived from a retrovirus and a nucleotide sequence that is absent in the retrovirus to prepare a mixture containing a viral component;
(b) preparing a double-stranded DNA derived from the recombinant retrovirus by adding deoxyribonucleotides to the mixture prepared in step (a) to form a complex that comprises the double-stranded DNA and a protein component derived from a retrovirus; and
(c) collecting the complex formed in step (b).

11. A method for producing a complex, the method comprising:
(a) isolating a double-stranded DNA containing a long terminal repeat derived from a retrovirus and a DNA having a nucleotide sequence that is absent in the retrovirus;
(b) isolating a protein component derived from a retrovirus;
(c) mixing the double-stranded DNA and the protein component derived from a retrovirus obtained in the previous steps together to form a complex; and
(d) collecting the complex formed in the previous step.

12. The method according to claim 11, wherein the protein component derived from a retrovirus is a culture supernatant of a packaging cell and/or an extract of a packaging cell.

13. The method according to any one of claims 8, 10 and 11, wherein the protein component derived from a retrovirus contains an enzyme having an activity of integrating a DNA having a long terminal repeat derived from a retrovirus into a chromosomal DNA.

14. A method for transferring a gene into a cell, the method comprising:
transferring, into a cell, a complex that comprises a double-stranded DNA containing a long terminal repeat derived from a retrovirus and a DNA having a nucleotide sequence that is absent in the retrovirus as well as a protein component derived from a retrovirus.

15. The method according to claim 14, wherein the complex is obtainable by the method defined by any one of claims 8, 10 and 11.

16. A method for transferring a gene into a cell, the method comprising:
transferring, into a cell, a protein component derived from a retrovirus as well as a double-stranded DNA containing a long terminal repeat derived from a retrovirus and a DNA having a nucleotide sequence that is absent in the retrovirus.

17. The method according to claim 16, wherein the protein component derived from a retrovirus is derived from a culture supernatant of a packaging cell.

18. The method according to claim 16, wherein the protein component derived from a retrovirus contains an enzyme having an activity of integrating a DNA having a long terminal repeat derived from a retrovirus into a chromosomal DNA.

19. A composition for transferring a gene into a cell, which contains a protein component derived from a retrovirus.

20. The composition according to claim 19, wherein the protein component derived from a retrovirus is forming a complex with a double-stranded DNA containing a long terminal repeat derived from a retrovirus and a DNA having a nucleotide sequence that is absent in the retrovirus.

21. The composition according to claim 20, wherein the complex is obtainable by the method defined by any one of claims 8, 10 and 11.

22. The composition according to claim 19, which further contains a vector having a long terminal repeat derived from a retrovirus.

23. The composition according to claim 22, wherein the vector is a vector for preparing a recombinant retrovirus.

24. The composition according to claim 22, wherein the vector is a plasmid vector.

25. The composition according to claim 19, wherein the protein component derived from a retrovirus is derived from a culture supernatant of a packaging cell and/or from an extract of a packaging cell.

26. The composition according to claim 19, wherein the protein component derived from a retrovirus contains an enzyme having an activity of integrating a DNA having a long terminal repeat derived from a retrovirus into a chromosomal DNA.

27. A kit for transferring a gene into a cell, which contains a composition for transferring a gene into a cell that contains a protein component derived from a retrovirus.

28. A kit for transferring a gene into a cell,
which is used for a method for transferring a gene into a cell,
which is in a packed form, and
which contains instructions that direct transferring, into a cell, of a complex that comprises a double-stranded DNA containing a long terminal repeat derived from a retrovirus and a DNA having a nucleotide sequence that is absent in the retrovirus as well as a protein component derived from a retrovirus.

29. A kit for transferring a gene into a cell,
which is used for a method for transferring a gene into a cell,
which is in a packed form, and
which contains instructions that direct transferring, into a cell, of a protein component derived from a retrovirus as well as a double-stranded DNA containing a long terminal repeat derived from a retrovirus and a DNA having a nucleotide sequence that is absent in the retrovirus.

30. A product of a reagent for transferring a gene into a cell,
which contains a packing material and a reagent for gene transfer enclosed in the packing material,
wherein the reagent for gene transfer contains a complex that comprises a double-stranded DNA containing a long terminal repeat derived from a retrovirus and a DNA having a nucleotide sequence that is absent in the retrovirus as well as a protein component derived from a retrovirus and/or a reagent for preparing the complex, and
wherein it is indicated in a label stuck to the packing material or instructions attached to the packing material that the reagent for gene transfer can be used for gene transfer that does not comprise a step of infecting a cell of interest with a recombinant retrovirus.

31. A product of a reagent for transferring a gene into a cell,
which contains a packing material and a reagent for gene transfer enclosed in the packing material,
wherein the reagent for gene transfer contains a protein component derived from a retrovirus, and
wherein it is indicated in a label stuck to the packing material or instructions attached to the packing material that the reagent for gene transfer can be used for gene transfer that does not comprise a step of infecting a cell of interest with a recombinant retrovirus.

32. The product according to claim 30 or 31, wherein the protein component derived from a retrovirus contains an enzyme having an activity of integrating a DNA having a long terminal repeat derived from a retrovirus into a chromosomal DNA.

33. A method for transducing a cell, the method comprising:
transferring, into a cell, a complex that comprises a double-stranded DNA containing a long terminal repeat derived from a retrovirus and a DNA having a nucleotide sequence that is absent in the retrovirus as well as a protein component derived from a retrovirus.

34. The method according to claim 33, wherein the complex is obtainable by the method defined by any one of claims 8, 10 and 11.

35. A method for transducing a cell, the method comprising:
transferring, into a cell, a protein component derived from a retrovirus as well as a double-stranded DNA containing a long terminal repeat derived from a retrovirus and a DNA having a nucleotide sequence that is absent in the retrovirus.

36. The method according to claim 35, wherein the protein component derived from a retrovirus is derived from a culture supernatant of a packaging cell and/or from an extract of a packaging cell.

37. The method according to claim 35, wherein the protein component derived from a retrovirus contains an enzyme having an activity of integrating a DNA having a long terminal repeat derived from a retrovirus into a chromosomal DNA.

38. A composition for transducing a cell, which contains a protein component derived from a retrovirus.

39. The composition according to claim 38, wherein the protein component derived from a retrovirus is forming a complex with a double-stranded DNA containing a long terminal repeat derived from a retrovirus and a DNA having a nucleotide sequence that is absent in the retrovirus.

40. The composition according to claim 38, wherein the complex is obtainable by the method defined by any one of claims 8, 10 and 11.

41. The composition according to claim 38, which further contains a vector for preparing a double-stranded DNA containing a long terminal repeat derived from a retrovirus and a DNA having a nucleotide sequence that is absent in the retrovirus.

42. The composition according to claim 41, wherein the vector is a vector for preparing a recombinant retrovirus.

43. The composition according to claim 41, wherein the vector is a plasmid vector.

44. The composition according to claim 38, wherein the protein component derived from a retrovirus is derived from a culture supernatant of a packaging cell and/or from an extract of a packaging cell.

45. The composition according to claim 38, wherein the protein component derived from a retrovirus contains an enzyme having an activity of integrating a DNA having a long terminal repeat derived from a retrovirus into a chromosomal DNA.

46. A kit for transducing a cell, which contains a composition for transducing a cell that contains a protein component derived from a retrovirus.

47. A kit for transducing a cell, which contains instructions that direct transferring, into a cell, of a complex that comprises a double-stranded DNA containing a long terminal repeat derived from a retrovirus and a DNA having a nucleotide sequence that is absent in the retrovirus as well as a protein component derived from a retrovirus for a method for transducing a cell.

48. A kit for transducing a cell,
which is used for a method for transducing a cell,
which is in a packed form, and
which contains instructions that direct transferring, into a cell, of a protein component derived from a retrovirus as well as a double-stranded DNA containing a long terminal repeat derived from a retrovirus and a DNA having a nucleotide sequence that is absent in the retrovirus.

49. A product of a reagent for transducing a cell,
which contains a packing material and a reagent for transduction enclosed in the packing material,
wherein the reagent for transduction contains a complex that comprises a double-stranded DNA derived from a recombinant retrovirus and a protein component derived from a retrovirus and/or a reagent for preparing the complex, and
wherein it is indicated in a label stuck to the packing material or instructions attached to the packing material that the reagent for transduction can be used for transduction that does not comprise a step of infecting a cell of interest with a recombinant retrovirus.

50. A product of a reagent for transducing a cell,
which contains a packing material and a reagent for transduction enclosed in the packing material,
wherein the reagent for transduction contains a protein component derived from a retrovirus, and
wherein it is indicated in a label stuck to the packing material or instructions attached to the packing material that the reagent for transduction can be used for transduction that does not comprise a step of infecting a cell of interest with a recombinant retrovirus.

51. The product according to claim 49 or 50, wherein the protein component derived from a retrovirus is an enzyme having an activity of integrating a DNA having a long terminal repeat derived from a retrovirus into a chromosomal DNA.

52. The method according to claim 14 or 16, wherein the gene is transferred into a eukaryotic cell.

53. The method according to claim 14 or 16, wherein the gene is transferred into a cultured cell derived from a eukaryotic cell.

54. The method according to claim 14 or 16, wherein the gene is transferred into a eukaryotic individual.

55. The method according to claim 14 or 16, wherein the gene is transferred into a vertebrate fertilized egg.

56. The method according to claim 14 or 16, wherein the gene is transferred into a vertebrate germ line.

57. The method according to claim 33 or 35, wherein a eukaryotic cell is transduced.

58. The method according to claim 33 or 35, wherein a cultured cell derived from a eukaryotic cell is transduced.

59. The method according to claim 33 or 35, wherein a eukaryotic individual is transduced.

60. The method according to claim 33 or 35, wherein a vertebrate fertilized egg is transduced.

61. The method according to claim 33 or 35, wherein a vertebrate germ line is transduced.
